(19) United States
(12) Patent Application Publication
(10) Pub. No.: US 2012/0215147 A1
(43) Pub. Date: Aug. 23, 2012

(54) Bunion belt big toe straightner

(76) Inventor: Marilyn Gay Lunnon, Jacksonville, FL (US)

(21) Appl. No.: 13/066,221

(22) Filed: Apr. 8, 2011

Related U.S. Application Data
(60) Provisional application No. 61/463,781, filed on Feb. 23, 2011.

(51) Int. Cl. A61F 5/00; A61F 5/14

(52) U.S. Cl. 602/30; 36/140

(57) ABSTRACT

The Bunion Belt Big Toe Straightner for use in correcting the alignment of the hallux including a section for surrounding the big toe; connected to an end brought across the bunion, tightened gently and fastened just below the lateral side of the bunion by the buckle; and in addition the belt or strap design can be manufactured into a shoe for everyday wear.

### CROSS REFERENCE TO RELATED APPLICATION

This application claims priority to and other benefit of U.S. Provisional Patent Application No. 61/463,781, filed Feb. 23, 2011, the entirety of which is incorporated by reference herein.

### BACKGROUND

Bunions, technically known as hallux valgus, are a set of signs and symptoms, where there is a certain degree of inward deviation of the first metatarsal, an outward deviation of the first toe (hallux), a deformity by rotation of the first toe, the lateral displacement of the sesamoids and tendon contractures (mainly of the adductor of the hallux and of the hallux and of the medial capsule (with respect to the exis of the foot).

The causes or factors responsible for bunions are diverse:

Inheritance—If there are precedents of bunions in a family, it is very likely that this predisposition will be inherited and hence there will be a high probability of developing them. Juvenile bunions, or that which arises during adolescence, usually has hereditary characteristics. Statistics prove that 63% of individuals with bunions have an immediate relative with bunions. Generally, bunions are caused by a hereditary defect in the mechanical structure of the foot. Bunions themselves are not hereditary; however, certain types of feet are more prone to develop bunions.

Footwear is one of the elements responsible for the appearance of bunions. There are studies that compare social groups that are always barefoot to social groups that use shoes.

Social groups that are permanently barefoot have a lower incidence of bunions. 33% of individuals that use shoes have bunions, in comparison to 2% of barefoot individuals.

Footwear has two kinds of effects: on one hand, it exerts pressure on the front part of the foot confining it to a reduced space and, and on the other hand, the toes move less than if barefoot, and this reduced mobility of the toes gives rise to an atrophy of the muscles of the feet. This decrease of strength in the muscles causes an imbalance in the forces that govern the direction of the toes.

Due to the above, bunions are very common for women between the ages of 40 and 60 who wear or have worn high heel shoes or shoes with a narrow tip. The fashion of narrow shoes that began after World War II caused an increase in this pathology. The foot is placed on an incline plane, on which it tends to be displaced forward. The front part of the foot experiences an overload; the toes hit against the shoe and shrink against the front tip. The posture of the skeleton of the foot is extended and laterally compressed, forcing the toes to cram. The whole foot is in a mold that deforms it position and molds it in this bad posture thorough compression.

When symptoms are not extreme, the patient may be concerned by the aesthetic aspects. The treatment of bunions must address the symptomatic aspects as well as the aesthetic aspects. Considering the general lengthy nature of this pathology and its possible hereditary and degenerative etiology, a conservative therapy should be initiated. Special footwear may be prescribed for bunions to make room for the deformity.

Bunions affect specifically 6-10% of the general population and it is the most frequent pathology of the foot, particularly among women (20% of adult women and 34% of elderly women).

Avoiding surgery is the first objective when seeking the relief of a person with bunions.

When hallux valgus is in its early stages, or where surgical correction is contraindicated, braces, straps, splints, orthotics, or combinations thereof may be used to manage progression of the deformity and relieve the associated symptoms.

Because braces, straps, splints, and orthotics are non-invasive, cost-effective, and may be self-applied, they represent important means of treating hallux valgus. These devices are typically designed to relieve the symptoms of the deformity by attempting to reduce the angle, realign the hallux, or combinations thereof by applying force to various portions of the foot. These devices may be used post-operatively to maintain appropriate alignment during the healing process, to prevent recurrence, or both.

Conventional non-surgical treatment devices have numerous disadvantages, including bulkiness, difficulty in wearing in shoes, requiring periodic readjustment, creation of undesired pressure and irritation, complexity of application, and failure to relieve the deformity. According, there is need for improved non-surgical devices or shoes in treating hallux valgus.

### SUMMARY

Currently there are no descriptions of leather belt-like braces, straps, splints, shoes or other orthotics for the prevention, treatment, and correction of the deformity that characterizes bunions.

The Bunion Belt Big Toe Straightner that constitutes the object of the present invention has the following advantages.

It is made like a leather belt or of any flaccid material with one end containing a buckle and the other end containing hooks or eyelets. One part of the belt fits around the big toe surrounding the hallux and separating it from the other toes.

The belt may wrap under the foot and around the heel to hold the belt in place and the other end gently pulls the big toe forward. The two ends are buckled like a belt straightening the big toe and gently pressing against the bunion or the metatarsal bone bringing it into proper alignment.

The belt is made of leather or other flaccid material for comfort and durability.

The belt or strap concept can be manufactured to fit into sandals or flip flops for all day wear.

Belt design with spaced eyelets allows the wearer to adjust the level of tension desired for big toe alignment.

Invention has a non-orthopedic look and can be made into different colors and types of leather.

It can prevent costly and painful surgery.

The Belt or Shoe can be used in the pre and post-surgical treatment of bunions. Moreover, it may be used preventively in cases of incipient hallux valgus, in patients who are genetically predisposed or for the comfort of the user.

### BRIEF DECRIPTION OF THE DRAWINGS

FIG. 1 Illustrates the unattached Belt. It shows the buckle, eyelets, and show how the invention would look if made without attachment to a shoe. Used alone, it would need to be wrapped around the big toe and the foot.

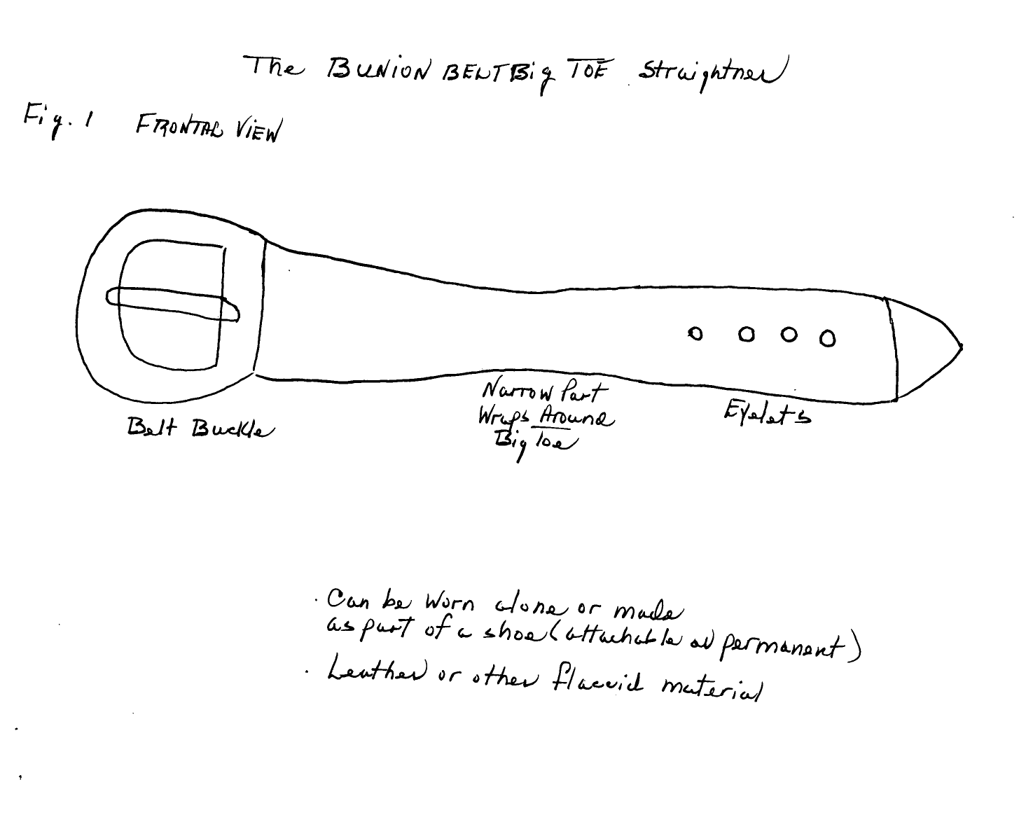

FIG. 2 Illustrates one example of how the Belt is worn and certain elements thereof

FIG. 3 An actual picture of a replica of the Belt wrapped around an actual toe of a patient with hallux valgus.

### DETAILED DESCRIPTION OF THE INVENTION

In accordance with the above, The Bunion Belt Big Toe Straightner, which is the object of the present application for a Patent of Invention, consisting of a leather belt-like design with one end containing a buckle and the other end hook or eyelets for fastening across the big toe.

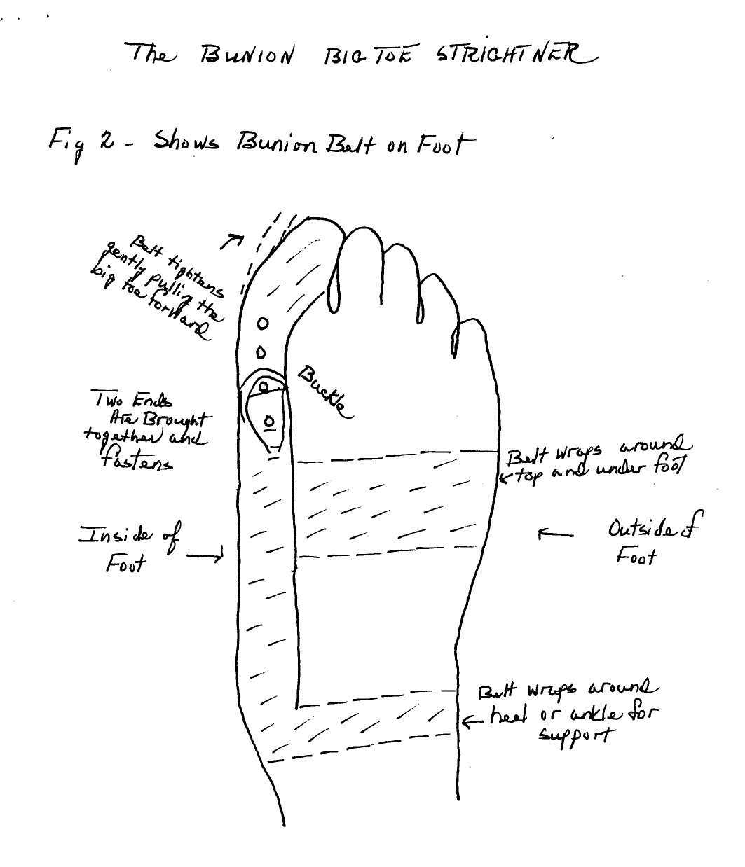

The first section (FIG. 2) of the Belt wraps around the big toe gently pulling the toe forward. One end is tightened against the bunion and buckled holding the big toe in place for alignment for both the big toe and forcing the bunion.

The first section (FIG. 2) allows separation of the first toe or hallux from the other toes or second section.

The second section (FIG. 2) wraps around the foot or is part of a shoe and is brought to meet the first section and buckled.

The material of the Belt or of the shoe is leather of any other flaccid material. Synthetic fibers can be used.

FIG. 2 illustrates that it can be made into a shoe and a leather portion wrapped across the front of the foot, around the heel, or the ankle for support. NOTE: The heel and sole of the shoe is not shown, only the frontal or top view.

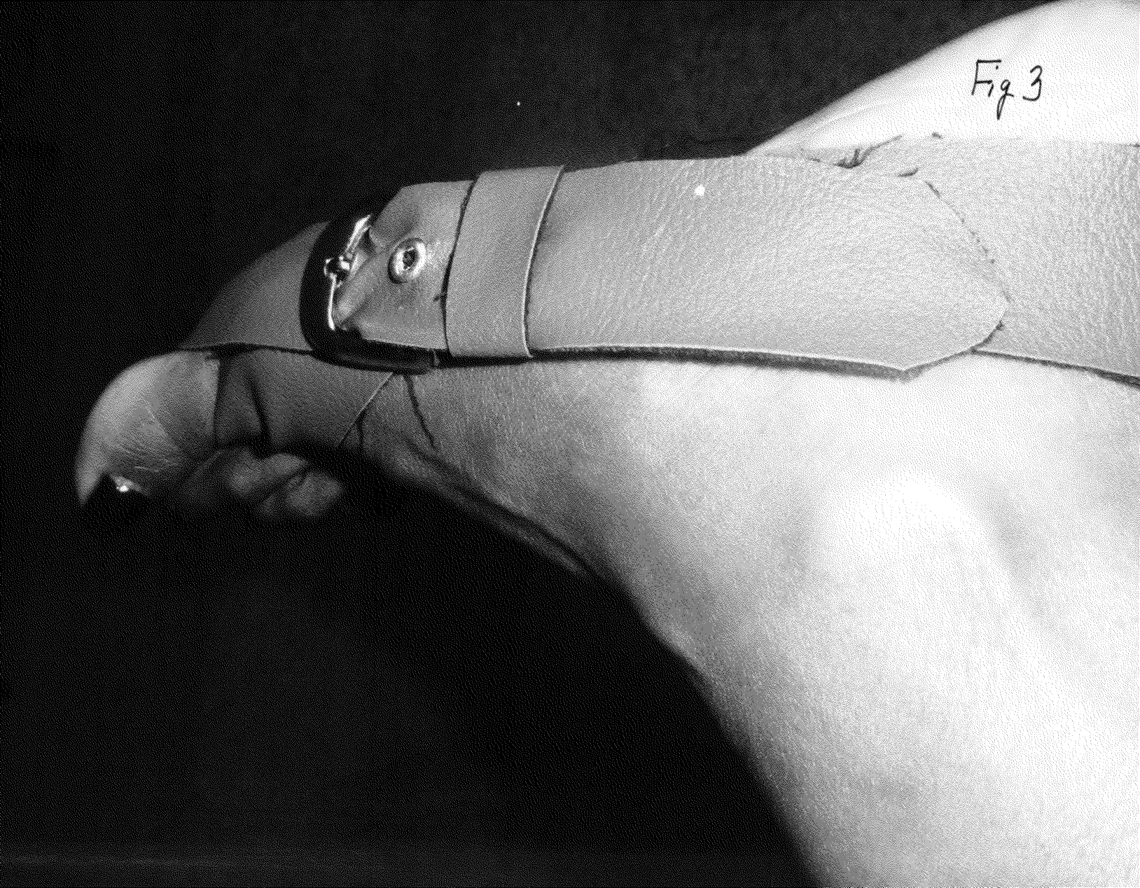

FIG. 3 is a photo that illustrates how the Belt separates the first toe or hallux from the other toes. It also illustrates how the Belt is buckled across the bunion with slight tension which in time will correct the condition.

The Belt can be used for the pre and post-surgical treatment of bunions and can be used preventively or for the comfort of the wearer.

The Belt is designed to be used alone, attached to a shoe, or manufactured into a shoe for men, women, and children in different models (lengths, colors, sizes, materials, and textures) with the possibility of being accompanied by other therapeutic or aesthetic properties.

1. A Belt, strap, or shoe for feet with bunions: comprising two ends with a section for inserting or wrapping around the big toe or hallux; one end has a buckle and the other end has eyelets for fastening across the lateral side of the bunion; made of leather or other flaccid material.

2. The Belt of claim 1, wherein the belt is manufactured from leather, synthetic, or any flaccid material or combination thereof.

3. The Belt or strap of claim 1, wherein is configured for use to be worn alone or manufactured as part of a shoe.

4. The Belt of claim 3, wherein the top of the shoe (except the heel and sole) is manufactured from the same material as the Belt—natural fibers, synthetic fibers, or any flaccid material.

5. The Belt of claim 1, wherein the belt is configured for being used for the pre and post surgical treatment of bunions.

6. The Belt of claim 3, wherein the shoe is configured to be used for pre and post surgical treatment of bunions.

7. The Belt of claim 1, wherein is configured for being used for the prevention and correction of incipient hallus valgus.

8. The shoe of claim 3, wherein is configured for being used for the prevention and correction of incipient hallus valgus.

9. The Belt of claim 1, wherein is configured for being used for patients who are genetically predisposed to hallus valgus.

10. The shoe of claim 3, wherein is configured for being used for patients who are genetically predisposed to hallus valgus.
